(19) United States
(12) Patent Application Publication
(10) Pub. No.: US 2004/0219695 A1
(43) Pub. Date: Nov. 4, 2004

(54) High sensitivity detection of and manipulation of biomolecules and cells with magnetic particles

(76) Inventor: John Stewart Fox, Encinitas, CA (US)

Correspondence Address: JOHN S. FOX, 684 POINSETTIA PARK SOUTH, ENCINITAS, CA, 92024, US

(21) Appl. No.: 10/347,963

(22) Filed: Jan. 21, 2003

Related U.S. Application Data
(60) Provisional application No. 60/350,635, filed on Jan. 19, 2002.

(51) Int. Cl. G01N 33/553

(52) U.S. Cl. 436/526

(57) ABSTRACT

The present invention generally relates to the field of biomolecule detection. More specifically, the present invention relates to compositions, methods and systems for the detection and manipulation of biomolecules using magnetic particles.

### CROSS-REFERENCE TO RELATED APPLICATION

This application claims priority based on U.S. provisional patent application No. 60/350,635 filed Jan. 19, 2002

### FIELD OF THE INVENTION

The present invention generally relates to the field of biomolecule detection. More specifically, the present invention relates to compositions, methods, and systems for the detection and manipulation of biomolecules and cells using magnetic particles.

### BACKGROUND OF THE INVENTION

In basic research, one goal is to understand how genes are distributed within populations and how expression of those genes leads to phenotypic differences. This information has the potential to become a powerful tool for predicting human health trends and has been a driving force behind the search for genetic markers for human disease.

Over the years, many biochemical techniques have been introduced for analyzing the presence and/or amount of a biomolecule in a sample. As examples, a number of organic stains have been adapted for the detection of electrophoretically separated proteins, including Bromphenol Blue, Coomassie Blue, Fast Green (Food Green 3) and Amido Black (Acid Black 1). (See Durrem, J. Am. Chem. Soc. 72:2943 (1950), Grassman and Hannig, Z Physiol. Chem. 290:1 (1952), Fazekas De St. Groth et al., Biochim. Biophys. Acta 71:377 (1963), and Meyer and Lamberts, Biochim. Biophys. Acta 107:144 (1965)). Fluorescent stains, such as fluorescamine and 2-methoxy-2,4-diphenyl-3(2H)-Furanone (MDPH), are also used to detect proteins (See Ragland et al., Anal. Biochem. 59:24 (1974) and Pace et al., Biochem. Biophys. Res. Commun. 57:482 (1974)). A sensitive technique for staining proteins is silver staining. (See Merril et al., Proc. Natl. Acad. Sci. USA 76:4335 (1979) and Switzer et al., Anal. Biochem. 98:231 (1979)). While these techniques may be useful to resolve total protein in a sample, they are limited in their usefulness to detect a specific protein in a heterogeneous population of proteins.

The detection of specific proteins in a sample can be accomplished by techniques including Western blot, immunoprecipitation, enzyme-linked immunoassay (ELISA), and sandwich assays. These techniques typically use radioactivity, fluorescence, and chemiluminescence to label or mark an antibody or other protein which binds to the target protein and thereby identifies the presence and/or location of the target. Depending on the quality of the antibody and the label used, the sensitivity of detection and non-specific binding varies.

Radioactivity, fluorescence, and chemiluminescence are also commonly used for the detection of specific nucleic acid sequences in a sample. Hybridization techniques, such as Southern and Northern blotting, are frequently employed to detect the presence of polymorphisms in a nucleic acid sample. In nucleic acid hybridization, for example, a radioactive label (e.g. ³²P or ³⁵S) is incorporated into an oligonucleotide probe which complements a target nucleic acid, and hybridization with the target is accomplished at a specific salt concentration and temperature. (See e.g. Sambrook, J. et al., Molecular Cloning, A Laboratory Manual (1989)).

Southern et al. has used nucleic acid-hybridization by setting up an array of oligonucleotides on plastic and glass, probing with a radioactive oligonucleotide, and detecting the presence of a target nucleic acid with a PhosphorImager. (See Southern, E. M. et al., Nucleic Acids Res 22, 1368-1373 (1994)). The PhosphorImager instrument, an expensive laser based optical system, and clean image-ready phosphor screens are needed for each sample read, making the system both cumbersome and very expensive. In addition, radioactive probes have a short shelf life (T₂=days to months) and require tight inventory control in a licensed facility. Although some companies are currently performing genetic screening using this method, the cost is prohibitive for most diagnostic procedures.

Others in the field are pursuing methods more predisposed to automation in hopes of enabling the rapid screening of a sample for a number of sequences. As one example, Affymetrix (Santa Clara, Calif.) has described a system which performs on-chip hybridization. (See Kreiner, T., American Laboratory March:39-43 (1996). In this system, oligonucleotides are arrayed in 90×90:m cells with 10⁷ oligonucleotides per cell, with 20,000 probe cells on each chip. This is annealed with fluorescence-labeled probes, and detection is carried out using a 488 nm Argon laser (8:m shot size) as a excitation source and a photomultiplier tube to detect the fluorescence emission. To read the chip, an optical system consisting of a dichromic mirror, scanning head, routing mirror and a confocal optical system are employed. One significant problem with this approach is non-specific background. Several natural occurring molecules either contribute to or quench the fluorescent signal, making this technique prone to a background noise which prevents this system from achieving highly sensitive nucleic acid detection.

Chemiluminescence is another marker employed to detect biomolecules. Chemiluminescence uses an enzyme coupled to the probe which catabolizes a chemical substrate to generate a photon. (See Bronstein, et al., BioTechniques 8:310-313 (1990)). Chemiluminescent nucleic acid hybridization assays may use a high performance, low-light-sensitive charge coupled device (CCD) camera to image the light emission from the chemical reaction. Often the camera is controlled by a personal computer and the images are archived on diskettes. While the CCD cameras are robust, CCD based systems do not have the sensitivity of film and the reagents have a one-year shelf life when stored at 4° C. (Tropix Inc.). As with fluorescence detection approaches, this approach is limited by background noise caused by naturally occurring enzymes or compounds contributing to the signal.

As the secrets of genomic regulation and the biosynthesis of enzymes, receptors, and ligands involved in human disease unfold, the need for detection techniques which provide a high degree of specificity and sensitivity with minimal background noise, while minimizing cost and handling issues, is manifest. In view of the foregoing, and notwithstanding the various efforts exemplified in the prior art, there remains a need for novel compositions, methods, and systems for highly sensitive biomolecule detection.

### SUMMARY OF THE INVENTION

Recognizing the limitations associated with current techniques for detection, manipulation and separation of biomolecules, the present invention provides methods and systems for the detection and manipulation of biomolecules and cells using magnetic particles. Through its embodiments, the present invention improves specificity and sensitivity while minimizing background, cost, time and handling issues related to biomolecule and cell detection and manipulation.

The present invention includes methods and systems, which use a magnetic moiety to external, manipulate internal cell process. The invention detects target molecules, molecular biomolecules or cells that have been contacted directly or indirectly with a magnetically labeled probe by subjecting the target-probe complex or cell to an applied magnetic field and determining the resulting magnetic characteristics. The invention provides methods and systems to prepare such magnetic probes or cells and to identify the presence and/or location of the target biomolecules disposed on a support or in solution or cells disposed on a support or in solution. The invention may detect characteristic responses of samples by several means, including but not limited to, by induced magnetization or orientation changes of magnetically labeled biomolecules.

The present invention includes highly sensitive biomolecule detection methods and systems, which use a magnetic moiety as a marker to determine the presence and/or location of a specific target biomolecules or cells. The invention allows magnetically tagged target molecules or molecular biomolecules to be added to the reaction at any time, and the addition of labeled magnetic particles to added at any time for enhancement of signal for magnetic detection of magnetically labeled biomolecules. The invention allows magnetically tagged cells or magnetic cells to be added to the reaction at any time, and the addition of labeled magnetic particles to added at any time for enhancement of signal for magnetic detection of magnetically labeled cells or magnetic cells.

The present invention also provides methods and systems for nucleic acid hybridization using magnetic labels, ferrofluids, and nonmagnetic colloids, as some examples.

The present invention also provides methods and systems to study binding and also provides methods and systems which use magnetic labels or magnetic cells to screen for, manipulate, and separate target cells, for example, in the same sample.

Methods and systems for the detection and separation of cells, as one example only, using ferrofluids and magnetic cells or magnetically tagged cells, are also provided. The invention also includes methods and systems to enhance the binding of a probe to a target biomolecules.

### BRIEF DESCRIPTION OF THE DRAWINGS

FIG. 1 displays a block diagram of an embodiment of a magnetic detection apparatus used to detect a magnetic particle joined to a biomolecule.

FIG. 2 is a graph of a hysteresis loop for a ferromagnetic material.

FIG. 3 is a graph of a portion of the hysteresis loops for neodymium iron boron and samarium cobalt.

FIG. 4 illustrates a typical output of an embodiment of a magnetic detection system in which a ferrofluid-labeled DNA sample was inserted into the reader at t=in and removed at t=out; the smooth line represents a 200 point running average.

FIG. 5 shows a semi-log plot of varying amounts of ferrofluid-labeled plasmid dsDNA (▪) or an oligonucleotide (O) spotted on a support and detected with an embodiment of a magnetic detection system; the relative magnetic unit reading (RMU) was the maximum voltage deflection of the spot corrected for background voltage and the line represents the mathematical fit to the data (equation is in the inset).

FIG. 6 shows a semi-log plot of varying amounts of ferrofluid-labeled RNA (▪) spotted on a support and detected with an embodiment of a magnetic detection system; the relative magnetic unit reading (RMU) was the maximum voltage deflection of the spot corrected for background voltage and the line represents the mathematical fit to the data (equation is in the inset).

### DETAILED DESCRIPTION OF THE PREFERRED EMBODIMENTS

The present invention comprises highly sensitive biomolecule or cell detection methods and systems which use a magnetic moiety as a marker to determine the presence, location, and/or quantity of a specific target biomolecules or cell, by way of examples only, a protein, lipid, tagged cell, magnetic cell, cells, or a nucleic acid, in a sample. Many types of magnetic labels may be joined to biomolecules or expressed in cells embodiments of the present invention. As examples only, preferred magnetic labels may include Fe³O⁴, Fe²O³, and rare-earth elements with atomic numbers between 64 and 69, inclusive, which have been incorporated into a colloidal suspension. In some preferred embodiments the magnetic labels are attached directly to the biomolecule, as one example only, a ferrofluid bound to a nucleic acid or protein, or in other embodiments, the magnetic label is indirectly attached to the biomolecule, e.g., through an intermediate, such as an antibody, a binding protein (e.g., avidin, streptavidin, and derivatives thereof), or a chemical linker.

Although attachment of a magnetic label, such as a ferrofluid, to a biomolecule disposed on a support is used in some embodiments of the present invention for the rapid identification of the presence, location, or quantity of a biomolecule, magnetically labeled biomolecules are also used as magnetic probes to specifically identify a target biomolecule which may be present in a heterogeneous sample of biomolecules. Accordingly, the invention provides methods and systems to prepare such magnetic probes and to identify the presence and/or location of the target biomolecules disposed on a support. One skilled in the art will appreciate that conventional approaches to nucleic acid hybridization and protein identification (e.g., immunoblotting and ELISA) are readily adapted to the magnetic detection methods and systems disclosed in preferred embodiments of the present invention. Furthermore, the present invention provides methods and systems to study competitive binding and techniques which enable the screening for several target biomolecules in the same sample.

Although attachment of a magnetic label, such as a ferrofluid, to a biomolecule disposed on a support is used in some embodiments of the present invention for the rapid identification of the presence, location, or quantity of a biomolecule, magnetically labeled biomolecules are also used as magnetic probes to specifically identify a target biomolecule which may be present in a heterogeneous sample of biomolecules. Accordingly, the invention provides methods and systems to prepare a cell to become magnetic by the addition of magnetic particles to the cell or the manipulation of genes that results in the expression of magnetic materials. One skilled in the art will appreciate that conventional approaches to gene expression (e.g., Green fluorescents proteins) are readily adapted to the magnetic materials production. The gene or genes that encode the production of magnetic materials or magnetic particles are expressed this is used for detection methods and manipulation systems disclosed in preferred embodiments of the present invention. In one embodiment, the present invention detects magnetically labeled cells by measuring or characterizing the magnetic signal generated by the magnetic particle in the cell. In several preferred embodiments, the invention uses a sensitive magnetic sensor, such as a giant magnetoresistive ratio sensor (GMR), for the detection of magnetic cells. A method of cellular detection according to one preferred embodiment of the present invention is as follows: A E. coli cell is injected with T4 Phage DNA from a bacteriophage. This DNA is tagged with a number of magnetic particles. This transfer of magnetically-labeled DNA makes the E. coli cell magnetic. The cell now has magnetic properties that can be detected, used to manipulate, and sort cells. But using different magnetic material to label the T4 Phage DNA and by characterizing the properties of a magnetically labeled biomolecule in an applied magnetic field, as one example only, by defining the hysteresis loop, solving one or more of the parameters of the hysteresis loop (e.g., saturation magnetization, remnant magnetization, and coercive force) or both, the identity as well as the quantity and location of the magnetic label are determined and there for the cell. Electroporation, and virus, proteins are method by which DNA or proteins that have magnetic tags can be moved in to cells and could be used in the above embodiment.

Furthermore by expressing the gene or genes that regulate the production of magnetic materials or magnetic particles in a cell that cell becomes magnetic. This would allow for the detection of the gene expression by a GMR detector or the manipulation of the cell by magnetic forces. How the gene or genes are expressed or which gene or genes are expressed the results of which is the production of different magnetic particles. These different magnetic particles have different magnetic signatures. The magnetic signature can are used to determine which genes are expressed or to identify the cell.

By characterizing the properties of a magnetic materials or magnetic particles in an applied magnetic field, as one example only, by defining the hysteresis loop, solving one or more of the parameters of the hysteresis loop (e.g., saturation magnetization, remnant magnetization, and coercive force) or both, the identity as well as the quantity and location of the magnetic materials or magnetic particles are determined.

The control of internal cell function or gene expression in this embodiment is controlled by the interaction of magnetic particles, magnetic labels and magnetic expression products with external magnetic fields, heat, light, pressure, electric fields and electromagnetic energy. In the approach the cells internal process are influenced by the interaction of the internal magnetic particles, which can be magnetic or nonmagnetic as a function of temperature and cause reactions to slow or speed up by magnetic pulsing. This approach would allow for a cell to be manipulated by external forces and driven in direction dictated by those forces. The regulated direction could be cell death, change in protein production and cell signals.

Additionally, the invention comprises methods and systems for the detection of one or more different cells in the same sample by using magnetically labeled DNA having different magnetic particles. Because many different magnetic particles exist and each has a unique magnetic signature, the detection of several different magnetically labeled biomolecules in the same sample is accomplished. Notably, the size and geometry of the magnetic particle affect magnetic characteristics, and therefore magnetic labels with homogeneous magnetic particles are preferred.

The invention also comprises methods and systems to enhance the binding of a probe to a target biomolecule and methods to reduce the background noise in hybridizations and binding assays. By applying a magnetic or electric field, or both, to regions of a support where a magnetically-labeled target biomolecule is disposed, for example, the movement toward and concentration of a magnetically labeled probe biomolecule near the region of the support having the target biomolecule is obtained. Advantages include improved binding kinetics and conservation of probe materials. Alternatively, a magnetic or electrical field, or both, is applied after a target biomolecule is bound by the magnetically labeled probe biomolecule so as to remove or separate from the magnetically-labeled target biomolecule and support any unbound or non-specifically bound magnetically labeled probe biomolecules. Further, the invention provides methods and systems by which magnetically-labeled cells are efficiently separated according to their magnetic potential, and in which magnetically-labeled cells in a solution are separated in an applied magnetic field. Because the amount of magnetically-labeled in the cell is directly related to the mass of the cell or it size or it cross-section, the invention comprises a magnetic-mass based separation technique in one embodiment.

Some preferred embodiments use types of magnetic labels. A “magnetic label” or “magnetic marker” is any transiently or permanently magnetized entity. In some embodiments of the present invention, a magnetic label comprises a magnetic particle that is ferromagnetic or ferrimagnetic or paramagnetic or superparamagnetic. The magnetic markers or labels preferably generate a magnetic signal, which can be, by way of example only, the magnetic field generated by ferromagnetic and ferrimagnetic materials, or the attraction for magnets characteristic of paramagnetic and superparamagnetic materials. In solution, the magnetic moments of the particles desirably align with each other.

In some embodiments, a magnetic label comprises a plurality of colloidal iron particles that define a respective magnetic moment. The term “magnetic labels” also refers to magnetic particles which comprise metal, metal compounds, or nuclei coated with a metal or metal compound or magnetic particles produced by the cell. In some embodiments, preferable magnetic labels include ferrofluids or other magnetizable colloids. Additionally, the term “magnetic label” refers to a magnetic particle including iron, cobalt, nickel, ferrous oxide, ferrous hydroxide, and other ferrous alloys, disposium oxide, and rare earth elements with atomic numbers between 64 and 69, inclusive, or magnetite (Fe₃O₄), maghemite (Fe₂O₃), and other mixed oxides. Magnetic labels having rare-earth magnetic particles are desirable because they may have a five-fold greater magnetization than iron oxide beads. The term “magnetic label” also refers to the magnets discussed in Vassiliou et al., J. Appl. Physics 73(10); 5109 (1993)), the disclosure of which is hereby incorporated by reference in its entirety.

One of ordinary skill in the art will appreciate that there are available biomolecule separation techniques that can be used prior to disposing a desired biomolecule on a support or used to separate and dispose the biomolecule on a support. There may be advantages for separating the desired biomolecule from other biomolecules present in a sample prior to contacting the sample with a magnetic label or a magnetically labeled probe biomolecule. Notably, the separation of the desired biomolecule often facilitates the isolation of the biomolecule after identification. The separation of the desired biomolecule from others in the sample is not necessary, however, to practice preferred embodiments of the present invention.

The present invention includes several methods and systems by which a target biomolecule can be disposed on a support in preparation for detection with magnetic labels or magnetically labeled probe biomolecules.

The separation of biomolecules prior to detection is accomplished, for example, by a one-dimensional or two-dimensional electrophoresis procedure. (See e.g., Methods in Enzymology Vol. 182, Guide to Protein Purification, ed. Deutscher, Academic Press Inc. pp. 425-477, San Diego, Calif. (1990), Current Protocols in Molecular Biology, Ausubel et al., ed., John Wiley & Sons (1994-1998), and Sambrook et al., Molecular Cloning: A Laboratory Manual, 2 ed., Cold Spring Harbor Laboratory, Cold Spring Harbor, N.Y. (1989)). Denaturing and non-denaturing gel electrophoresis are frequently used to separate nucleic acids, and sodium dodecyl sulfate polyacrylamide gel electrophoresis (SDS/PAGE) is a common method to separate proteins. Further, pulse-field electrophoresis, two-dimensional protein electrophoresis, isoelectric focussing, and other separation techniques are used to separate target biomolecules prior to detection with a magnetic label or a magnetically labeled probe. Additionally, biomolecules can be separated chromatographically, for example, by thin layer chromatography (TLC), by liquid chromatography techniques, such as high performance liquid chromatography (HPLC) or fast performance liquid chromatography (FPLC), or by affinity chromatography techniques, prior to detection with a magnetic label or a magnetically labeled probe.

Another common laboratory technique called “blotting” is also used to dispose a target biomolecule on a support. This technique allows for the transfer of separated biomolecules on a matrix to a solid membrane or a filter. (See e.g., Current Protocols in Molecular Biology, Ausubel et al., ed., John Wiley & Sons (1994-1998), and Sambrook et al., Molecular Cloning: A Laboratory Manual. 2 ed., Cold Spring Harbor Laboratory, Cold Spring Harbor, N.Y. (1989)). Additionally, biomolecules disposed on a membrane support by blotting are frequently immobilized or fixed into position so that further rounds of detection can be accomplished. By “stripping” or removing the first bound probe by techniques known in the art, subsequent rounds of detection with new magnetically labeled probes are performed.

In preferred embodiments, the present invention may include a “matrix” or “support” which may be a carrier, a bead, a resin, or any macromolecular structure used to attach, join, immobilize, or dispose thereon a biomolecule, by way of examples only, a nucleic acid, lipid, or protein. Supports may include, but are not limited to, the walls of wells of a reaction tray, test tubes, polystyrene beads, fluorescent beads, magnetic beads, nitrocellulose strips, membranes, microparticles such as latex particles, sheep (or other animal) red blood cells, cells, fluorescent particles, duracytes® and others. Additionally, organic carriers including proteins and oligo/polysaccarides (e.g. cellulose, starch, glycogen, chitosane or aminated sepharose) and inorganic carriers such as silicon oxide material (e.g. silica gel, zeolite, diatomaceous earth or aminated glass) may be used in embodiments of the present invention. Furthermore, in some embodiments, a liposome or lipid bilayer (natural or synthetic) may be used as a support. Desirable supports may also include polyacrylamide gels, agarose gels, composite gels, and other gel matrices, papers, chips, membranes, chromatography matrices, as used in thin layer chromatography, and resins or beads, as used in affinity chromatography.

In some embodiments of the present invention, the support has a hydrophobic surface that interacts with a portion of the biomolecule by hydrophobic non-covalent interaction. As one example only, the hydrophobic surface of the support is oftentimes a polymer such as plastic or any other polymer in which hydrophobic groups have been linked, such as polystyrene, polyethylene or polyvinyl. In some embodiments, the support has a charged surface which interacts with the biomolecule, as one example only, a charged nitrocellulose or nylon membrane. In other embodiments, the support is attached to a biomolecule through a linker, such as biotin-avidin or biotin-streptavidin, or biotin and an avidin or streptavidin derivative. The supports used in some embodiments of the present invention have other reactive groups which are chemically activated so as to attach a biomolecule. As some examples, cyanogen bromide activated matrices, epoxy activated matrices, thio and thiopropyl gels, nitrophenyl chloroformate and N-hydroxy succinimide chlorformate linkages, and oxirane acrylic supports art are adapted for use in some embodiments.

In the present invention, any type of biomolecule, by way of examples only, proteins, polypeptides, nucleic acids, and lipids, can be joined or disposed on a support and subsequently joined to a magnetic label. Further, preparations of biological samples having biomolecules can be joined or disposed on a support. In preferred embodiments, several different biomolecules or different preparations of biological samples having biomolecules or cells are attached to a support in an ordered array wherein each biomolecule, cell or preparation of biological sample is attached to a distinct region of the support which does not overlap with the attachment site of any other biomolecule or preparation of biological sample. Preferably, such an ordered array is designed to be “addressable” where the distinct locations are recorded and can be accessed as part of an assay procedure.

In some embodiments, addressable biomolecule arrays comprise a plurality of different biomolecule probes that are joined to a support in different known locations. The knowledge of the precise location of each biomolecule probe makes these “addressable” arrays particularly useful in binding assays. As one example only, an addressable array can comprise a support joined to many different antibodies that recognize different human proteins that are tumor markers for various forms of cancer. The proteins from a preparation of biological sample from a human subject are magnetically labeled (e.g., using a ferrofluid labeling process, discussed below), and the magnetically labeled sample is applied to the array under conditions that permit antibody binding. If a protein in the sample is recognized by an antibody on the array, then a magnetic signal will be detected at a position on the support that corresponds to the antibody-protein complex. Since each antibody and its position on the array are known, an identification of the protein/tumor marker and, thus, the disease state of the subject, are rapidly determined. Additionally, one embodiment can employ nucleic acid probes joined to a support to form an array of magnetically labeled nucleic acids from a biological sample from a human subject. In this manner, by way of example, disease prognosis may be assessed based on the use of nucleic acid probes which are associated with sequences that have been associated with human disease and the detection of magnetically labeled complementary nucleic acid sequences present in the biological sample. These approaches are easily automated using technology known to those of skill in the art of high throughput diagnostic analysis.

The present invention may comprise in its embodiments any addressable array technology known in the art. One embodiment of polynucleotide arrays is known as the Genechips™, and has been generally described in U.S. Pat. No. 5,143,854; PCT publications WO 90/15070 and 92/10092. These arrays may generally be produced using mechanical synthesis methods or light directed synthesis methods, which incorporate a combination of photolithographic methods and solid phase oligonucleotide synthesis. (See Fodor et al., Science, 251:767-777, (1991)). The immobilization of arrays of oligonucleotides on solid supports has been rendered possible by the development of a technology generally identified as “Very Large Scale Immobilized Polymer Synthesis” (VLSIPS™) in which, typically, probes are immobilized in a high-density array on a solid surface of a chip. Examples of VLSIPS™ technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

Preferred embodiments of the present invention include several methods and systems to detect the magnetic signal of a magnetic label that is attached to a biomolecule. Although many biological molecules incorporate iron, biological materials generally exhibit no net magnetic field. (See Stryer, L., Biochemistry (1993)). Accordingly, the magnetic signal generated by a magnetic label attached directly or indirectly to a biomolecule is accurately measured and characterized with a high degree of sensitivity and little background noise.

For embodiments that simply detect the presence of a magnetic label on a support (and, hence, whether the biomolecule is attached to a magnetic label), a magnetic sensor such as an inductive read head (e.g., the read head used in a Toshiba model KT-53 stereo cassette) can be used. In contrast, when a relatively precise measurement of the strength of the magnetic field generated by the magnetic label is desired to ascertain not simply the presence of the magnetic label attached to a biomolecule on a support but also the location of the biomolecule, the sensor is desirably a magnetoresistive (MR) read head, as examples only, the read heads used in certain existing disk drives and/or the MR heads made by IBM of Armonk N.Y. or Eastman Kodak Co. of Rochester N.Y. and disclosed by Smith et al. in J App. Physics 69(8):5082 (1991). In some embodiments, the invention uses a MR sensor that is embedded in a chip, wherein the chip has a surface to accommodate the deposit of biomolecules or cells. In other embodiments, the sensor may be a magnetic force microscope, SQUID sensor, metal film Hall-effect device, or a ultra-high sensitivity susceptometer (for sensing paramagnetic and superparamagnetic markers), such as the device disclosed by Slade et al. in IEEE Transactions on Magnetics, 23 (5):3132 (1992).

In one embodiment, a sensitive giant magnetoresistive ratio sensor on a solid-state chip (“GMR Sensor”) is used in a magnetic detection system to identify the presence of a magnetic label attached to a biomolecule. The GMR sensor, which may run on very low wattage, is a rugged solid-state chip which is mass produced inexpensively. The utility of the GMR based sensor is highlighted in one respect by its sensitivity to small changes in a nearby magnetic field. GMR materials exhibit an order of magnitude greater sensitivity to changes in magnetic field strength than standard anisotropic magnetoresistive materials and saturate at larger fields yielding an improved dynamic range. (See Daughton, J. et al., IEEE Trans. Mag 30 (1994), Barnas, J. et al. PhysiRevi B 42:8110(1990)).

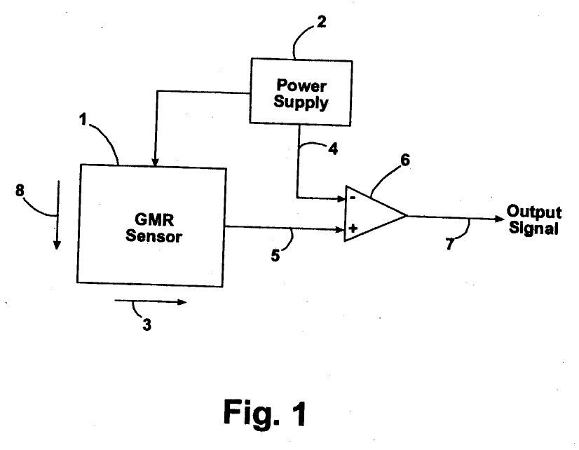

A GMR sensor may be used in the configuration illustrated in FIG. 1. A commercially available sensor 1 (model T15, Nonvolatile Electronics Inc., Eden Prairie Minn.) is coupled to a power supply 2. The sensor 1 advantageously includes biasing magnets for producing an applied biasing magnetic field 3. The input voltage on line 4 and the output of the sensor on line 5 are routed to an operational amplifier 6, and the output signal 7 is measured. This output signal 7 will vary with variations in the intensity of an externally applied magnetic field 8.

A generous dynamic range is obtained by the GMR sensor because the final voltage output depends on the sensitivity and range of the GMR sensor chip, the applied magnetic field, and the input voltage. That is, for any given GMR chip both the offset magnetic bias and the input voltage are easily manipulated to allow for a wide range of detection sensitivity.

GMR materials may be composed of alternating 15-40 Å layers of ferromagnetic metals such as CoFe, NiFeCo and alloys such as CuAgAu. To make a sensitive magnetic sensor, the GMR materials may be etched into four resistors on a chip hooked together in a Wheatstone bridge with two of the resistors shielded from magnetic fields. When an external magnetic field is applied, the resistance of the two unshielded GMR material resistors changes and unbalances the bridge. When an input voltage is put across the bridge, the output voltage increases with the application of a magnetic field. The output voltage is desirably read directly or, for small applied fields, the voltage deviation from the offset voltage is amplified with a commercially available op-amp, as illustrated in FIG. 1. The output voltage is preferably displayed using a digital oscilloscope program running on a personal computer, for example. A desirable detection system is disclosed in the published PCT application having International Publication No. WO 96/05326 to Fox, the disclosure of which is incorporated by reference herein in its entirety.

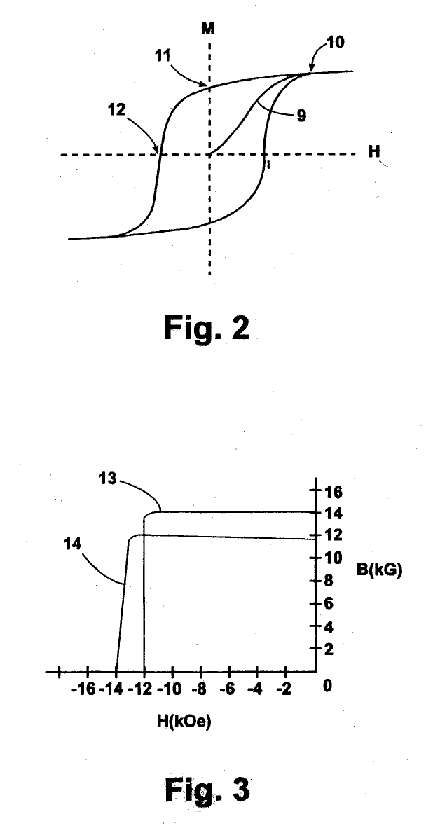

In one embodiment of the invention, the measured magnetic signal is used not only to determine the presence of magnetic label but also to distinguish between different magnetic labels. One of ordinary skill in the art will appreciate generally that different magnetic materials have different magnetic properties. In addition to the fundamental classes of magnetic behavior mentioned above, such as paramagnetism, diamagnetism, ferromagnetism, and the like, different materials within each class have distinguishable magnetic characteristics. For example, ferromagnetic materials exhibit hysteresis in the presence of a varying applied magnetic field. This is illustrated in FIG. 2, which shows a graph of magnetization (M) versus applied magnetic field intensity (H) for a hypothetical ferromagnetic material. If an unmagnetized sample of ferromagnetic material is subjected to an increasing applied magnetic field intensity, the magnetization of the material will increase along line 9 of FIG. 2. As the field strength is increased, the material reaches a saturation magnetization 10 and no further magnetization takes place as the applied field is increased. Following saturation, if the applied field is slowly reduced, the magnetization of the material will also be reduced. Upon return to zero applied field, however, a remnant magnetization 11 will remain. If the direction of the applied field is then reversed and increased slowly from zero in the opposite direction, the remnant magnetization will be reduced as the material begins to re-orient in the new direction of the applied magnetic field. The applied field strength required to eliminate the remnant magnetization so that the material is demagnetized is known as the coercive force 12. If the field is increased still further in the opposite direction, the material will become increasingly magnetized in that direction, until saturation is again reached, but in the opposite direction. Reducing the field to zero again results in a remnant magnetization of the same magnitude as the first, but of the opposite polarity. This process of magnetization under an applied field defines a “hysteresis loop” that is characteristic of the material. The three parameters of the hysteresis loop described above, saturation magnetization, remnant magnetization, and coercive force, are each different for different types of ferromagnetic material, and thus, magnetic probes or labels made from different types of material may be distinguished based on these differing magnetic properties.

The present invention also includes methods and systems to assess ratios of these parameters. For example, the ratio of remnant magnetization to the saturation magnetization is known as the “remnant squareness” of the hysteresis loop. The slope of the M-H curve when the hysterisis loop crosses zero magnetization (i.e., at the coercive force designated 12 in FIG. 2) is also a characteristic of the material. Another parameter known as “loop squareness” approaches 1 as the hysteresis curve at this point becomes increasingly vertical. These other parameters derived from the hysteresis curve may be especially useful in differentiating magnetic labels, as the measured numerical value of a ratio of measurements or a rate of change of magnetization can be less dependent on the concentration of label in the sample being measured.

In FIG. 3, a graph illustrating magnetization as a function of applied field intensity is provided for two different materials. This graph shows the upper left quadrant of the hysteresis curve for neodymium iron boron 13, and for samarium cobalt 14. It can be seen from examination of this Figure that the neodymium iron boron material has a higher saturation magnetization, lower coercive force, and steeper slope at zero magnetization. These features may be used to distinguish the presence of magnetic labels made from different materials.

Embodiments of the present invention measure the magnetization of a selected sample material as a function of applied magnetic field strength. From these measurements, aspects of the hysteresis loop exhibited by the sample are determined. Three types of equipment frequently used to characterize the magnetic properties in materials are the 60-Hz M-H looper, the toroidal B-H looper, and the vibrating sample magnetometer (VSM). Any of these commercially available instruments, or other comparable equipment or systems, can be used to measure magnetic properties of labels. Therefore, a sample containing a magnetic label of a first kind is distinguished from a sample containing a magnetic label of a second kind. Typically, and as illustrated by FIG. 3, the different labels will have different chemical composition. For example, they can comprise two different iron alloys, or an iron based label and a rare earth element based label. Samples containing these labels will exhibit hysteresis loops having different shapes, and are thus distinguishable with magnetization analysis under an applied external magnetic field. Mixtures of two different labels are also detectable because the sample will exhibit a hysteresis loop having characteristics that are intermediate between the loops exhibited by the two labels individually.

The present invention also includes methods and systems to detect and characterize magnetic labels or magnetic cells attached to a biomolecule disposed on a support. Because magnetic labels attached to a biomolecule or magnetic cells generate a quantifiable magnetic field, the presence and location of biomolecule or magnetic cells on the support can be determined. For example, in one preferred embodiment, when a support having a biomolecule attached to a magnetic label is juxtaposed with a magnetic sensor and moved past the magnetic sensor, the magnetic field of the attached magnetic markers variably permeate the sensor and thereby cause the sensor to generate a detection signal. This same approach would be used to detect signal from magnetic cells. For label characterization, an external magnetic field is applied, and sample magnetization is measured at a plurality of applied field strengths. When detecting the presence of label, the support is preferably closely juxtaposed with the sensor and, more preferably, the substrate is distanced from the sensor, by way of examples only, by only a few microns or less, so as to improve the sensitivity of detection. In other embodiments, the support and the sensor may be integrated. The sensor is electrically connected to a signal processor that receives the detection signal and generates a signal representative of the amount of magnetic label present. This same approach could be used to detect magnetic cells.

The signal processor includes signal processing circuitry known in the art for processing signals from magnetic sensors, as well as a correlator for generating a biomolecule concentration based upon the detection signal from the magnetic sensor. For example, the correlator can be a programmable chip or a microprocessor having software, which interprets the magnetic signal information to calculate and display biomolecule concentration. Desirably, the correlator is calibrated to generate accurate biomolecule concentration by means well-known in the art, e.g., by passing several supports having known quantities of a biomolecule deposited thereon next to the sensor and adjusting the resulting detection signals to the known concentrations. Additionally, the signal from the signal processor can be sent to an output device.

If desired, the present invention may also include a transporter and a support that can be positioned on the transporter to move the support past the sensor. In one embodiment, the sensor is moved past the substrate in a raster-scan type motion to generate a two-dimensional data output, e.g., an image, having an “x” dimension and a “y” dimension. Further, the two-dimensional data output can be transformed into a three-dimensional output wherein the third dimension (“z” dimension) represents magnetic signal intensity.

Preferred embodiments of the invention provide methods and systems to attach a biomolecule with a detectable magnetic label. In some embodiments, a biomolecule is directly attached to a magnetic label (e.g., by an interaction with a ferrofluid) and in others the biomolecule is indirectly attached to a magnetic label (e.g., by an interaction with a magnetically-labeled protein, as described below). Positively charged ferrofluids offer many advantages over other types of magnetic labels. These ferrofluid magnetic labels have no special storage, handling or disposal requirements and are relatively easy to fabricate. Ferrofluids are commercially available and ferrofluids having many different types of magnetic particles and, thus, different magnetic properties, can be custom-made and obtained through Ferrofluidics, Nashua, N.H. Each particle in a ferrofluid has an intrinsic magnetic moment that can be aligned and accentuated with the application of an external orienting field. In solution, ferrofluids exist in a colloidal state but when ferrofluids bind to a biomolecule their collodial properties diminish. Additionally, ferrofluids in a colloidal state are not strongly attracted by a magnet, however, when bound to a biomolecule, the magnetic properties of a ferrofluid permit magnetic attraction. A ferrofluid's loss of colloidal properties upon binding to a biomolecule and the ability to attract biomolecules bound to a ferrofluid with a magnet are exploited by the invention to separate ferrofluid-bound biomolecules from biomolecules and ferrofluid which have not interacted.

In some embodiments, a ferrofluid is attached to a biomolecule (e.g., a probe for the detection of a specific nucleic acid sequence or protein domain) and separated from unbound biomolecules and unbound ferrofluids in many ways. In one embodiment, a biomolecule is contacted with a ferrofluid for a time sufficient to allow the magnetic label to interact with the biomolecule. Subsequently, centrifugation is performed to loosely pellet the biomolecules having attached magnetic labels. The supernatant is removed and the pellet is resuspended in distilled water or a suitable buffer. This “washing” procedure is desirably performed several times so as to effectively remove all the unbound ferrofluid and unbound biomolecules. The unbound colloidal ferrofluid and unbound biomolecules remain in solution, while the ferrofluid bound to the biomolecule is pelleted and, thus, separated from the unbound ferrofluid and unbound biomolecules.

In another embodiment, a magnet is used to separate biomolecules bound to a ferrofluid from unbound ferrofluids and unbound biomolecules. As above, a biomolecule is contacted with a ferrofluid for a time sufficient to allow the magnetic label to interact with the biomolecule. Subsequently, a magnet is applied, for example, to the side of the vessel housing the biomolecule and ferrofluid, and the biomolecules having attached magnetic labels are aggregated near the magnet. The supernatant is carefully removed and the magnetic aggregate is resuspended in distilled water or a suitable buffer. This “washing” procedure is desirably performed several times so as to effectively remove all the unbound ferrofluid and unbound biomolecules. The unbound colloidal ferrofluid and unbound biomolecules remain in solution, while the ferrofluid bound to the biomolecule is aggregated and, thus, separated from the unbound ferrofluid and unbound biomolecules.

As indicated above, embodiments of the present invention include the use of positively charged ferrofluid colloids to directly label a probe biomolecule. Such techniques can also be used in the invention to label a biomolecule disposed on a support so as to detect its presence and location. The detection of a biomolecule disposed on a support, for example, is accomplished by applying the ferrofluid to the biomolecule, washing away unbound or non-specifically bound ferrofluid, and detecting the magnetic signal generated by the bound magnetic label. From the information generated by the magnetic signal from the support, the presence and location of the biomolecule are determined.

The invention also includes methods and systems to attach a magnetic label to a target biomolecule indirectly by binding a magnetically labeled secondary molecule to the target biomolecule. In addition to the use of magnetically labeled probe biomolecules to detect specific sequences or proteins, as will be discussed below, magnetically labeled secondary molecules, as examples only, nucleic acids or proteins, are used to detect biomolecules disposed on a support. As examples, and without limitation, in some embodiments a magnetic label is attached to a nucleic acid which interacts with a protein binding domain such as found in transcription factors or other nucleic acid binding proteins. In other embodiments, a magnetic label is attached to a protein which interacts with a modified nucleotide within a nucleic acid sequence or a modified domain of a protein. In the latter instance, magnetically labeled antibodies specific for modified biomolecules, such as dinitrophenol (DNP), isopentenyl-6-adenosine (I₆A), and biotin, are used. Additionally, biotin residues on a nucleic acid or protein are readily detectable with embodiments that use magnetically labeled avidin, streptavidin, monomeric avidin, and derivatives or modifications of these proteins. Accordingly, these proteins are preferably labeled with a ferrofluid and are separated from unbound protein and ferrofluid by the methods detailed above, however, several commercially available magnetic antibodies and magnetic avidin and streptavidin are available.

Embodiments of the invention also include methods and systems to detect specific biomolecules within a population of heterogeneous biomolecules disposed on a support. One of ordinary skill in the art will appreciate that many conventional approaches to specific nucleic acid detection, such as Northern and Southern hybridization, and specific protein detection, such as Western blotting and immunoprecipitation, are adaptable for use with embodiments of the present invention. In some embodiments of the present invention, a non-magnetic colloid or other blocking agent which binds to single stranded nucleic acid or non-specific binding sites on a target biomolecule are added. Non-magnetic colloids, such as silver stain, and blocking agents, such as Salmon sperm DNA, carrier RNA, bovine serum albumin, ovalbumin, and casein, are added to reduce non-specific binding of probes and background noise.

One embodiment of the invention identifies a specific biomolecule (e.g., proteins or nucleic acids) within a population of heterogeneous biomolecules is as follows: First, a sample having a target biomolecule, among a heterogeneous population of biomolecules, is disposed on a support. The target biomolecule on the support is then contacted with a magnetically-labeled probe biomolecule that interacts with the target biomolecule. The unbound and nonspecifically bound magnetic probe is removed by washing in a suitable buffer, and the bound magnetic signal is measured and characterized with a magnetic sensor, as described above. Accordingly, the presence of a magnetic signal at a specific location on the support identifies the presence of the target biomolecule. Alternatively, as discussed above, one or several different probe biomolecules can be disposed on a support at different locations so as to create an addressable array that is used to detect the presence of one or more target biomolecules in a preparation of biological sample. Magnetically labeled biomolecules present in the biological sample are applied to the array, the support is washed so as to remove unbound and nonspecifically bound biomolecules, and the magnetic signal that remains on the support is detected using a magnetic sensor, and the presence of the target biomolecule in the biological sample is identified.

In other embodiments, many different probes or biological samples or both are screened at the same time. By using a method referred to as “multiplexing”, the invention screens biomolecules present in several biological samples, including samples from different individuals, against a battery of probe biomolecules in the same reaction to determine predispositions to disease, genetic typing, and forensic identification, as examples.

In one embodiment, an addressable array is constructed wherein many different probe biomolecules (e.g., nucleic acid probes or antibodies or other types of protein probes) are disposed on a support at locations that are separate from one another and readily identifiable. The locations and identities of the probe biomolecules on the support are recorded (e.g., on a recordable computer media such a computer disk, hard drive, CD ROM, DVD ROM, or other recordable media as known in the art). Biological samples from three individuals, for example, having biomolecules that correspond or are detectable by probes on the array if the target biomolecule is present are obtained and prepared, according to conventional techniques in hybridization or blotting or both. The three different biological samples are separately labeled with different magnetic labels (e.g., ferrofluids) such that the first is labeled. The magnetically labeled biological samples are washed so that only specifically bound magnetically labeled biomolecules remain in the samples and the samples are pooled.

The pooled sample now comprises the biomolecules of three different individuals and three different magnetic labels. The pooled sample is then contacted to the array under conditions which allows for specific binding of the probe biomolecules to any target biomolecules that may be present in the three different samples. The unbound and nonspecifically bound biomolecules are removed by washing in a suitable buffer, and the array is passed before a magnetic sensor which characterizes and measures the magnetic signals bound to the support in an applied magnetic field, for example. Because each of the three different magnetic labels has a magnetic particle that has a unique magnetic signal (e.g., hysteresis curve shape and slope, saturation magnetization, remnant magnetization, coercive force, etc.), the identity of the presence or absence of each type of magnetic particle can be accomplished in the same reaction. Thus, the detection of one, two, or three magnetic signals from one or more locations on the array can be accomplished using this embodiment of the present invention, and the ability to rapidly screen several individuals for many different indicators for disease and genetic composition has been accomplished.

Gene expression uses a number of methods for determining if a gene is activated. A number of bacteria use magnetism as part of their life cycle. This includes the production of magnetic particle, this is production is regulated by gene that encode the production of select proteins to manufacture magnetic iron compounds. The magnetic iron compounds that are manufactured have a unique magnetic signature. One of ordinary skill in the art will readily recognize embodiments of the multiplexing method of the invention can be used to screen a number individual cells each with a unique magnetic signature. As different genes are expressed that encode for the production of magnetic particles a different unique magnetic signature is defined allowing the monitoring of genes. One benefit is as the gene is expressed the cell becomes magnetic and as such can be magnetically manipulated. Because each of the three different magnetic cells has a magnetic particle that has a unique magnetic signal (e.g., hysteresis curve shape and slope, saturation magnetization, remnant magnetization, coercive force, etc.), the identity of the presence or absence of each type of magnetic particle produced by the cell can be accomplished in the same reaction. This approach could be an array of cells as described in the above embodiment.

The hybridization reaction is commonly performed in liquid. The unique approach is to do a dry hybridization, the reaction is performed with dried down DNA in a dry environment. The DNA can be tagged with a magnetic practical this would allow for the movement and manipulation of the DNA fragments in the dry environment. A pulsing magnetic system can be used to speed the reaction and present the biomolecules for hybridization. This approach could be used with a wide number of biomolecules or cells.

One embodiments of the present invention include a lock and key approach with the magnetically-labeled probe biomolecules acting as the key and a GMR sensor as the lock. The magnetic tags size is such that it closes a magnetic loop the effect is an increase in signal single event detection. The probe biomolecules is bound to the GMR chip such that when the magnetically-labeled probe biomolecule gene bind to it then close a gap in the flux collector closing the magnetic flux loop. The GMR chip senses this as an event. And addition embodiment the magnetically-labeled probe biomolecules would short out the conductive layer between the two GMR layers the effect would be to have the two layer interact this would cause a large swing in the resistance and hence the signal. A cell or cells made magnetic by the methods described with in, could be used in this embodiment.

Additional embodiments may include, as examples only, fluorescent cells which produce fluorescent signal (for example, like green fluorescent protein), and produce magnetic particles or magnetic materials could be monitored by a CCD camera the movement of a cell producing a fluorescent signal in the field of view of an optical device, such as a spectrophotometer or CCD camera image in a applied magnetic field would be a indication of both magnetic and fluorescent expression. The cell magnetic properties could be used to sort or capture it.

The invention includes methods and systems to enhance the binding of a probe biomolecule to a target biomolecule and to reduce non-specific binding and background noise. In one embodiment, a target biomolecule (e.g., a nucleic acid or protein) is disposed on a support and is contacted with a probe biomolecule having an attached magnetic label, as described above. To enhance binding, a magnetic field is applied to regions of the support near the target biomolecule so as to induce the magnetically labeled probe biomolecule to move toward and concentrate at the position corresponding to the target biomolecule. In this manner, a greater binding to the probe biomolecule is obtained. Additionally, an electric field is applied in conjunction with the magnetic field so as to enhance the movement toward and concentration at the site near the disposed target biomolecule. In another embodiment, an electrical field or a magnetic field or both are applied to the support after binding of the target biomolecule by the magnetically-labeled probe so as remove or separate from the target biomolecule any unbound or non-specifically bound probe biomolecule.

In some embodiments, a pulsing electrical or magnetic field is used to move the probe biomolecule toward the target biomolecule and concentrate it at that site or, alternatively, to induce the unbound probe biomolecule or non-specifically bound probe biomolecule to move away from the target biomolecule. By applying the approaches described above, a magnetically labeled probe can be concentrated at a site near the target biomolecule and thereby increase the kinetics of binding, and unbound and non-specifically bound probe can be separated from the specifically bound probe so as to reduce background.

The invention includes methods and systems to enhance the binding of a probe biomolecule to a target biomolecule and to reduce non-specific binding and background noise. In one embodiment, a target biomolecule (e.g., a nucleic acid or protein) is disposed on a support and tagged with magnetic particles and is contacted with a probe biomolecule having an attached magnetic label, as described above. To enhance binding, a magnetic field is applied to regions of the support near the target biomolecule since the target biomolecules have magnetic tags this produces a higher magnetic field gradient at the location of the biomolecules/magnetic tags complex so as to induce the magnetically labeled probe biomolecule to move toward and concentrate at the position corresponding to the target biomolecule. In this manner, a greater binding to the probe biomolecule is obtained. Additionally, an electric field is applied in conjunction with the magnetic field so as to enhance the movement toward and concentration at the site near the disposed target biomolecule. In another embodiment, an electrical field or a magnetic field or both are applied to the support after binding of the target biomolecule by the magnetically-labeled probe so as remove or separate from the target biomolecule any unbound or non-specifically bound probe biomolecule. In the above embodiment, with the addition of static or pulsing vibration to improve kinetic of binding or evenness of binding over a surface.

In some embodiments, a pulsing electrical or magnetic field is used to move the probe biomolecule toward the target biomolecule and concentrate it at that site or, alternatively, to induce the unbound probe biomolecule or non-specifically bound probe biomolecule to move away from the target biomolecule. By applying the approaches described above, a magnetically labeled probe can be concentrated at a site near the target biomolecule and thereby increase the kinetics of binding, and unbound and non-specifically bound probe can be separated from the specifically bound probe so as to reduce background. In the above embodiment, with the addition of static or pulsing vibration to improve the kinetic of binding or evenness of binding over a surface.

Preferred embodiments of the invention may separate magnetically labeled biomolecules on the basis of mass, size by applying a magnetic field and charge by labeling. In one embodiment, the invention provides methods and systems that separate magnetically labeled biomolecules according to their mass in an applied magnetic field. Biomolecules are first labeled with a magnetic marker, preferably a ferrofluid, for example by the approaches detailed above. Once the biomolecules are magnetically labeled, they are suspended in a solution (e.g., a suitable buffer) and a magnetic field is applied to the sample. Because the amount of ferrofluid which binds to the biomolecule is directly proportional to the mass of the biomolecule, molecules with greater mass have a greater magnetic potential than smaller molecules. Accordingly, magnetically labeled biomolecules are separated according to their mass by applying a strong magnetic field. Magnetic labeled biomolecules are moved by a strong magnetic field to a screen or screens of a defined sized, the screen will stop biomolecules to large to migrate and allow smaller biomolecules to continue. By using a label that binds by ionic charge only that charge biomolecules will become magnetic. The methods described in the above embodiments employing magnetic static and pulsing fields, electric static and pulsing fields, static and pulsing vibrations would be used in the sorting process.

In some embodiments, the type and class of magnetic labels can influence the signal produced. A recent development in particle research is nanorods, there are magnetic materials produced in the shape of a rod. This magnetic rod could be attached to biomolecule by using method and chemistry used to attach other labels. An additional approach is to use biomolecules or metal that intercalates with the DNA molecule, like Ethidium bromide. In the case of metals that intercalates the attachment of metal-to-metal presents fewer problem that metal to organics.

As pointed out in early embodiments a pulsing electrical or magnetic field is used to move the probe biomolecule toward the target biomolecule and concentrate it at that site or, alternatively, to induce the unbound probe biomolecule or non-specifically bound probe biomolecule to move away from the target biomolecule. By applying the approaches described above, a magnetically labeled probe can be concentrated at a site near the target biomolecule and thereby increase the kinetics of binding, and unbound and non-specifically bound probe can be separated from the specifically bound probe so as to reduce background. An additional feature of the approach would to be add a vibration both static and changing to the pulsing and static approach described in early embodiments.

As pointed out in early embodiments a pulsing electrical or magnetic field is used to move the probe biomolecule toward the target biomolecule and concentrate it at that site or, alternatively, to induce the unbound probe biomolecule or non-specifically bound probe biomolecule to move away from the target location. By applying the approaches described above, a magnetically labeled probe can be concentrated at a target location. In a processed called magnetic self-assembly the concentrated biomolecules are linked to the surface by chemical linkers, or physical attachments. The chemistry can be always active or activated by light, head, or electromagnetic energy allowing the bonding to the surface the concentrated target biomolecule. An additional feature of the approach would to be add a vibration both static and changing to the pulsing and static approach described in early embodiments. A cell made magnetic by the methods described with in, could be used in this embodiment.

One embodiment is to use a controlled magnetic field to hold magnetically labeled biomolecules or magnetic beads out of a reaction so they can be added at a later time. This could control the reaction, enhance the signal or probe targets.

An additional embodiment is a new approach to hybridization. The samples are allowed to bind in a dry environment. The DNA is magnetically labeled and died down the died probe DNA is placed on a surface that has target DNA, the pulsing magnetic field and vibration system described above is employed to manipulate the probe DNA to interact and bind to target DNA. This allow for very small volumes to be used and a faster reaction times. Cells and proteins made magnetic by the methods described with in, could be used in this embodiment.

The final packaging of GMR sensors in most cases includes a flux concentrators, the flux concentrator funnels the magnetic flux to the GMR sensor. The shape and material that make up of the flux concentrator can enhance the transfer of flux from the sample to the sensor. It can also allow the sample to be some distance from the sample with out a large lost in signal. The invention would employ flux concentrators that would interact with the sample by taking into account the size and shape of the sample. This would enhance the signal or allow the sample to be some distance from the GMR sensor or both. This allow for a more flexible design and signal enhancement.

The following examples are provided for exemplary purposes and are not intended to limit embodiments of the present invention.

### EXAMPLE 1

The probe nucleic acid is made by incubating 2 ug of a complementary oligonucleotide, T55, with ferrofluid (1:1 (v:v) in 10:1 total volume). Unbound T55 is separated and removed from the ferrofluid conjugated T55 by washing with water, as described above.

In this example, the present invention is the placement of nucleic acid on a support by means of a magnetic label attached to a nucleic acid. In this example, the invention uses a magnetic label attached to a nucleic acid probe As an example, a solution with a number of oligonucleotide of 52 nucleotides (T54) nucleic acid is made by incubating 2 ug of a oligonucleotide, T54, with ferrofluid (1:1 (v:v) in 10:1 total volume), distilled water is added a strong magnet is applied to pull down the ferrofluid conjugated oligonucleotides. The unbound ferrofluid stays in solution the ferrofluid conjugated oligonucleotides are pulled to the magnetic and out of solution. The unbound ferrofluid and unbound T55 is separated and decanted from the ferrofluid conjugated T55 that was pulled down by magnetic. This is repeated until the decanted fluid is clear.

A solution that contains oligonucleotide (T54) conjugated with ferrofluid is placed on the glass slide with its surface prepared for the linking of DNA. At several locations a magnetic pull down forced is applied such that the pulled down DNA is concentrated into a small dot. The result of the magnetic pull down is a concentration for the oligonucleotide (T54) at the locations where the magnetic force was applied. The DNA is covalent bonded to the glass surface.

### EXAMPLE 2

A solution that contains oligonucleotide (T54) conjugated with ferrofluid is placed on the glass slide with its surface prepared for the linking of DNA. At several locations a magnetic pull down forced is applied such that the pulled down DNA is concentrated into a small dot. The result of the magnetic pull down is a concentration for the oligonucleotide (T54) at the locations where the magnetic force was applied. At the location of the DNA concentration a pulse of UV light is applied covalent bonding the DNA to the glass surface.

### EXAMPLE 3

A solution that contains oligonucleotide (T54) conjugated with ferrofluid is placed on the glass slide with its surface prepared for the linking of DNA. At several locations a magnetic pull down forced is applied such that the pulled down DNA is concentrated into a small dot. The result of the magnetic pull down is a concentration for the oligonucleotide (T54) at the locations where the magnetic force was applied. At the location of the DNA concentration heat is applied this heat is a required step for the bonding of the DNA to the glass surface. The glass heated by the underlying electrical heater.

### EXAMPLE 4

A solution that contains oligonucleotide (T54) conjugated with ferrofluid is placed on the glass slide with its surface prepared for the linking of DNA. At several locations a magnetic pull down forced is applied such that the pulled down DNA is concentrated into a small dot. The result of the magnetic pull down is a concentration for the oligonucleotide (T54) at the locations where the magnetic force was applied. At the location of the DNA concentration heat is applied this heat is needed for the bonding of the DNA to the glass surface. The magnetic particles are headed by absorption of IR light; this heats the DNA fragment and its binding site. Allowing the DNA to bind to the glass surface.

### EXAMPLE 5

A solution that contains oligonucleotide (T54) conjugated with ferrofluid is placed on the glass slide with its surface prepared for the linking of DNA. At several locations a magnetic pull down forced is applied such that the pulled down DNA is concentrated into a small dot. The result of the magnetic pull down is a concentration for the oligonucleotide (T54) at the locations where the magnetic force was applied. At the location of the DNA concentration heat is applied this heat is needed for the bonding of the DNA to the glass surface. The magnetic particles are headed by absorption of microwaves, this heat the DNA fragment and its binding site. Allowing the DNA to bind to the glass surface.

### EXAMPLE 6

The invention also detects a target nucleic acid by first using a biotinylated ferrofluid tagged nucleic acid probe and a then a streptavidin beaded. In this example, the invention uses a biotinylated ferrofluid tagged nucleic acid probe in conduction with the magnetic kinetics technology to speed the reaction and the streptavidin bead to enhance the signal for detection. Magnetic label to identify the presence and location of a biotinylated nucleic acid probe hybridized to a target nucleic acid disposed on a support. The probes for these experiments comprise a DNA-biotin-streptavidin-magnetic bead complex. Biotinylated 40-mer oligonucleotides complementary to a regions of the 8 phage genome are used. There are many custom service companies for oligonucleotide synthesis but, desirably, the nucleic acid probes are made on the premise using a Milligen Cyclone Plus synthesizer at the 0.012 micromole scale. Commercially available modification chemicals are used to quantitatively biotinylate the oligonucleotide directly on the synthesis column (Cruachem). There are also several commercial sources of streptavidin magnetic beads (MPG, Dynal, Promega or Boehringer Mannheim). In order to reproduce an optimum coupling efficiency, various dilutions of the 5 um beads will be contacted with the biotinylated oligonucleotide. Desirably, the highest magnetic concentration is sought so as to minimize the possibility that any given bead will have more than one oligo attached. Custom preparations of magnetic beads, having a single streptavidin molecule per bead, are also obtainable. (Bangs Labs, Fishers, Ind.).

DNA from 8 phage is isolated and a region encoding the D gene is used as the target nucleic acid. (See Mikawa et al., J. Mol. Biol. 262:21 (1996) for a description of suitable target nucleic acid sequences and complementary probe nucleic acid sequences). There are a number of well-known chemicals used to isolate viral RNA or DNA. (Sambrook, J. et al., Molecular Cloning, A Laboratory Manual 1989)). Phenol, for example, denatures the coat proteins of virus and liberates the nucleic acids inside. The proteins aggregate at the phenol/water interface and the nucleic acid remains in the aqueous phase. Phenol is, however, a moderately caustic chemical and several methods that rely on less harsh agents have been developed. Numerous formulations based on combinations of detergents (SDS, SLS, Nonidet P40, reductants (DTT and beta-ME), proteases (Proteinase K, pronase) and chaotropics (guanidine, guanidinium thiocyanate) have also been published. (Sambrook, J. et al., Molecular Cloning, A Laboratory Manual (1989), Luria, S. E. et al., General Virology and Calendar, R., The Bacteriophages (1988)). Once the phage DNA is isolated, it is spotted at various concentrations on a nylon membrane and crosslinked with a standardized dose of UV light (1200 units in a Stratalinker). The filter is also, preferably, prehybridized with a non-magnetic colloid and/or a blocking agent. The filter is then brought to 6×SSC and the magnetically-labeled probe (biotinylated ferrofluid tagged nucleic acid probe) is added. Hybridization is conducted using magnetic kinetics (magnetic kinetics uses a pulsing or static magnetic field to concentrate the probe near the target, speeding the kinetics of the hybridization reaction) at 20° C. below the calculated Tₘ. After washing, the streptavidin magnetic beads are added an allowed to bind with the biotinylated DNA that hybridized. The addition of the magnetic beads is used to enhance the signal, sample is measured for magnetic activity with a GMR sensor, as described above. The addition of the beads can be controlled electromagnetically.

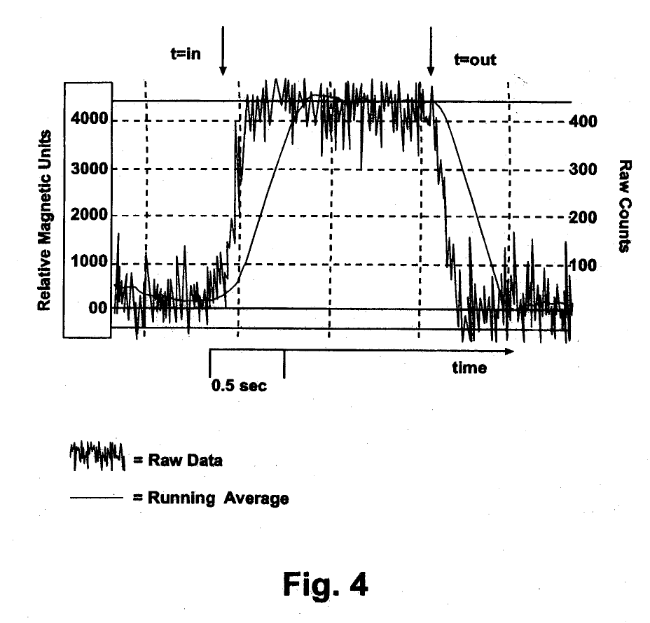

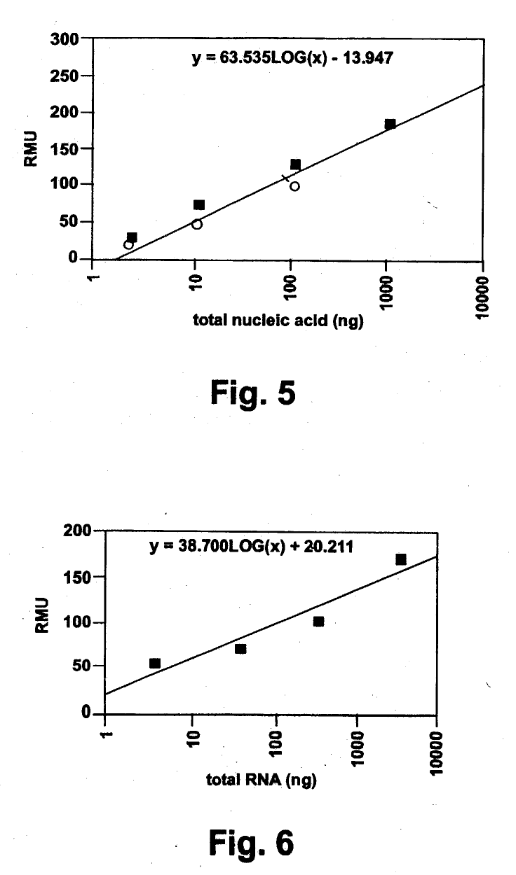

Samples are measured using a GMR sensor (Ti 5 model; Nonvolatile Electronics Inc., Eden Prairie Minn.) with bias-magnets, and the voltages are recorded by a PC after analog processing and 12 bit A/D conversion. Output from the prototype magnetic detection system unit for the detection of nucleic acids is shown in FIG. 4. The magnetism of the DNA/ferrofluid filters is also measured with a vibrating sample magnetometer (VSM, Digital Measurement Systems Inc.) to verify and calibrate the results. The VSM determines the actual emu generated at the surface of the sample, whereas the GMR sensor determines the relative emu. Triplicate samples, prepared identically to the one used to generate the GMR sensor data shown in FIG. 4, yield an average of 4.5×10³ (±0.7) emu. From this data it may be determined that one relative magnetic unit (RMU) equals≈10⁵ emu.

### EXAMPLE 7

The invention also detects a target nucleic acid by using a biotinylated nucleic acid probe and a ferrofluid-labeled streptavidin marker. In this example, the invention uses streptavidin conjugated with a magnetic label to identify the presence and location of a biotinylated nucleic acid probe hybridized to a target nucleic acid disposed on a support. The probes for these experiments comprise a DNA-biotin-streptavidin-magnetic bead complex. Biotinylated 40-mer oligonucleotides complementary to a regions of the 8 phage genome are used. There are many custom service companies for oligonucleotide synthesis but, desirably, the nucleic acid probes are made on the premise using a Milligen Cyclone Plus synthesizer at the 0.012 micromole scale. Commercially available modification chemicals are used to quantitatively biotinylate the oligonucleotide directly on the synthesis column (Cruachem). There are also several commercial sources of streptavidin magnetic beads (MPG, Dynal, Promega or Boehringer Mannheim). In order to reproduce an optimum coupling efficiency, various dilutions of the 5 um beads will be contacted with the biotinylated oligonucleotide. Desirably, the highest magnetic concentration is sought so as to minimize the possibility that any given bead will have more than one oligo attached. Custom preparations of magnetic beads, having a single streptavidin molecule per bead, are also obtainable. (Bangs Labs, Fishers, Ind.).

DNA from 8 phage is isolated and a region encoding the D gene is used as the target nucleic acid. (See Mikawa et al., J. Mol. Biol. 262:21 (1996) for a description of suitable target nucleic acid sequences and complementary probe nucleic acid sequences). There are a number of well-known chemicals used to isolate viral RNA or DNA. (Sambrook, J. et al., Molecular Cloning, A Laboratory Manual 1989)). Phenol, for example, denatures the coat proteins of virus and liberates the nucleic acids inside. The proteins aggregate at the phenol/water interface and the nucleic acid remains in the aqueous phase. Phenol is, however, a moderately caustic chemical and several methods that rely on less harsh agents have been developed. Numerous formulations based on combinations of detergents (SDS, SLS, Nonidet P40, reductants (DTT and beta-ME), proteases (Proteinase K, pronase) and chaotropics (guanidine, guanidinium thiocyanate) have also been published. (Sambrook, J. et al., Molecular Cloning, A Laboratory Manual (1989), Luria, S. E. et al., General Virology and Calendar, R., The Bacteriophages (1988)).

Once the phage DNA is isolated, it is spotted at various concentrations on a nylon membrane and crosslinked with a standardized dose of UV light (1200 units in a Stratalinker). The filter is also, preferably, prehybridized with a non-magnetic colloid and/or a blocking agent. The filter is then brought to 6×SSC and the magnetically-labeled probe (biotinylated oligonucleotide bound to magnetically-labeled streptavidin) is added. Hybridization is conducted for 16 hours at 20° C. below the calculated T. After washing, the sample is measured for magnetic activity with a GMR sensor, as described above. As a negative control, 8 DNA is spotted on the filter and hybridized with a non-complementary oligonucleotide coupled to a magnetic bead. As a positive control, a biotinylated oligonucleotide probe is labeled with a streptavidin-alkaline phosphatase conjugate and the filter is developed with standard precipitating substrates. These results demonstrate that nucleic acid hybridization using magnetically-labeled streptavidin molecules bound to biotinylated nucleic acid probes can be accomplished.

### EXAMPLE 8

The invention also uses nucleic acid hybridization that exploits the magnetic signal generated by a ferrofluid. In this example, the invention uses the increase in magnetic signal obtained by a nucleic acid hybrid over a single stranded nucleic acid to identify the presence and location of the nucleic acid hybrid. In this example, a first oligonucleotide of 52 nucleotides (T54) is used as a target nucleic acid and a second complementary oligonucleotide (T55), is used as a probe nucleic acid. One microliter of the target nucleic acid T54 (at 0.5 ug/ml) is spotted on a nylon membrane and is crosslinked to the membrane with UV light (autolink setting; Stratalinker 2500; Stratagene). Subsequently, the membrane is washed briefly with distilled water and is allowed to air dry. The unlabeled probe nucleic acid (T55) is suspended in 3 ml of a 1×SSC solution (Sambrook, J. et al., Molecular Cloning, a Laboratory Manual, (1989)).

The support having the target nucleic acid (T54) and the 3 ml of 1×SSC solution containing the unlabeled probe nucleic acid (T55) are combined in a 15 ml conical tube. The hybridization is conducted in an oven at 50° C. for 16 hrs. The negative control for the experiment is run in parallel, and uses T55 as both the probe and target nucleic acid. The experimental and control filters are removed from the oven, washed in 1×SSC, and air dried. Next, the supports are placed in a 15 ml conical tube containing a 3 ml suspension of ferrofluid (1:1 v/v). Binding of the ferrofluid to the nucleic acids present on the supports is conducted for 5 minutes. The supports are then removed and washed in 1×SSC and air dried. The magnetic signal present on the supports is then determined using a GMR sensor, as described above.

The sample having the T54 target nucleic acid and the T55 probe will have a greater average RMU than the sample having the T55 target nucleic acid and the T55 probe. Furthermore, another washing of the membrane with a lower salt concentration (e.g., 0.1 SSC) will promote strand displacement and a decrease in signal for the support having the T54 target nucleic acid will be observed. These results demonstrate that a conventional nucleic acid hybridization with an unlabeled probe can be performed and sensitive detection of nucleic acid hybrids, by using a ferrofluid after hybridization, can be accomplished.

### EXAMPLE 9

The invention also detects several target cells in a sample by using multiple magnetically labeled probes. In this example, the invention identifies multiple target cells in a biological sample having polynucleotides in the same reaction by using multiple magnetically labeled nucleic acid probes. For example, a first cells magnetic particles or labels are made of hematite Fe2O3 the second cells magnetic particles or labels are made of magnetite Fe3O4. The first cell has a magnetic particle that is different, (therefore having a different magnetic characteristic), from the second cells magnetic particle. For example, the magnetic particles for the two cells is made of different iron or transition metal alloys having different hysteresis characteristics. It will be appreciated that more than two magnetic particle can be used, in accordance with this embodiment, to detect multiple cells present in a biological sample, so long as each cell has a different magnetic particle.

Then a magnetic signal which corresponds to the first and/or the second magnetic label will be detected by analyzing, for example, the magnetic hysteresis characteristics of the biological sample. Thus, rapid diagnostic screening using multiple cells having different magnetic labels can be accomplished.

Preferred embodiments of the present invention have been disclosed. A person of ordinary skill in the art would realize, however, that certain modifications would come within the teachings of this invention, and the following claims should be studied to determine the true scope and content of the invention. In addition, the methods and structures of the present invention can be incorporated in the form of a variety of embodiments, only a few of which are described herein. It will be apparent to the artisan that other embodiments exist that do not depart from the spirit of the invention. Thus, the described embodiments are illustrative and should not be construed as restrictive. All references cited herein are hereby expressly incorporated by reference.

What is claimed is:

1. A method of identifying the presence of magnetically labeled cell in a sample comprising the steps of:
providing a sample containing one or more cells;
contacting the target cell with a magnetic label under conditions which permit the formation of a cell-label complex;
subjecting said sample to an applied magnetic field; and
detecting a characteristic response of said sample to an applied magnetic field.

2. The method of claim 1, wherein said characteristic response is defined at least in part by an induced magnetization of said sample.

3. The method of claim 1, wherein said characteristic response is defined at least in part by an induced orientation change of said magnetically labeled cells.

4. A method for cell detection comprising the steps of:
providing a cell,
contacting the cell with a magnetic probe under conditions which permit the formation of a cell magnetic probe hybrid.
identifying the presence of the cell by detecting the magnetic characteristic of the magnetic probe.

5. The method of claim 4, where the cell is it solution.

6. The method of claim 4, where the cell is disposed on a support.

7. The method of claim 4, where the magnetic probe is inside the cell

8. The method of claim 4, where the magnetic probe is produced inside the cell by the cell.

9. The method of claim 4, where the magnetic probe is produced inside the cell by gene expression.

10. The method of claim 4, where the magnetic probe is placed inside the cell by phage.

11. The method of claim 4, where the magnetic probe is placed inside the cell by electroporation.

12. A method of assembly of biomolecule comprising the steps of:
providing a target biomolecule disposed on a support;
providing a probe biomolecule comprising a magnetic label;
contacting the target biomolecule with the probe biomolecule, wherein the probe biomolecule interacts with the target biomolecule; and
applying a magnetic field to the target biomolecule and the probe biomolecule such that the probe biomolecule is induced to move toward the disposed target biomolecule and bind to the target biomolecule.
Repeating these steps to produce chains of biomolecules

13. A method according to claim 12, wherein the probe biomolecules magnetic labels are removed after binding.

14. A method according to claim 12, wherein the bound magnetically labeled biomolecules are manipulated by a magnetic field to from structures.

15. A method according to claim 12, wherein the magnetic label is attached to the probe biomolecule by a linker to be manipulated by a magnetic field to from structures.

16. A method for assaying molecules in a sample comprising the steps of:
providing a sample which contains one or more target molecules or molecular complexes;
contacting said target with one or more probes under conditions which permit the formation of a target-probe complex, wherein the probe comprises one or more magnetic labels;
subjecting said target-probe complex to an applied magnetic field; and
determining one or more magnetic characteristics of said target-probe complex wherein said sensing means comprises a giant magnetoresistive ratio sensor and flux concentrator with a flux gap; and
the target-probe magnetic label closes the flux gap

17. The method of claim 16, Wherein said sensing means comprises a giant magnetoresistive ratio sensor and conductive layer gap; and the target-probe magnetic label closes the conductive layer gap.

18. A method for assaying molecules in a sample comprising the steps of:
providing a sample which contains one or more target molecules or molecular complexes;
contacting said target with one or more probes under conditions which permit the formation of a target-probe complex, wherein the probe comprises one or more magnetic labels;
subjecting said target-probe complex to an applied magnetic field; and
determining one or more magnetic characteristics of said target-probe complex wherein said sensing means comprises a giant magnetoresistive ratio sensor and flux concentrator.
Wherein the flux concentrator is a cone shape with the small end attached to the giant magnetoresistive ratio sensor and the larger end in close proximity to the target-probe complex.

19. The method of claim 18, Wherein the flux concentrator is a rod shape with one end attached to the giant magnetoresistive ratio sensor and the other end in close proximity to the target-probe complex

20. The method of claim 18, Wherein the flux concentrator one end attached to the giant magnetoresistive ratio sensor and the other end in close proximity to the target-probe complex from an addressable array

21. A method of enhancing the binding of a probe biomolecule to a target biomolecule comprising the steps of:
providing a target biomolecule disposed on a support;
providing a probe biomolecule comprising a magnetic label;
contacting the target biomolecule with the probe biomolecule, wherein the probe biomolecule interacts with the target biomolecule; and
applying a magnetic field to the target biomolecule and the probe biomolecule such that the probe biomolecule is induced to move toward the disposed target biomolecule.
applying a vibration static or changing frequency to the target biomolecule and the probe biomolecule such that the probe biomolecule is induced to move toward the disposed target biomolecule.

22. The method of claim 21, wherein the magnetic field is applied in a pulsing fashion.

23. The method of claim 21, wherein the reaction is carried out dry
